(19) United States
(12) Patent Application Publication
(10) Pub. No.: US 2009/0169646 A1
(43) Pub. Date: Jul. 2, 2009

(54) METHODS OF TREATING CYSTIC FIBROSIS

(75) Inventors: Gregory Bosch, Malvern, PA (US); Claire Sampson, Doylestown, PA (US)

Correspondence Address: COOLEY GODWARD KRONISH LLP;ATTN: Patent Group, Suite 1100, 777 - 6th Street, NW, WASHINGTON, DC, 20001, US

(73) Assignee: PURICORE, INC., Malvern, PA (US)

(21) Appl. No.: 12/280,486

(22) PCT Filed: Feb. 22, 2007
(86) PCT No.: PCT/US07/62594

§ 371 Date: Jan. 15, 2009

Related U.S. Application Data
(60) Provisional application No. 60/775,362, filed on Feb. 22, 2006.

(51) Int. Cl. A61K 33/00

(52) U.S. Cl. 424/661

(57) ABSTRACT

A method of treating and/or managing cystic fibrosis in a patient by administering an electrolyzed saline solution containing hypohalous acid. Preferably, the hypohalous acid is hypochlorous acid. Also provided is a method of treating and/or managing cystic fibrosis by administering an electrolyzed saline solution at specific pHs such as between 5.0 and 6.2. The present invention also provides a method of administering an electrolyzed saline solution for treating and/or managing cystic fibrosis using a combined electrochemical generator and a vaporizer, humidifier or fogger. Combination therapies are also provided.

### FIELD OF THE INVENTION

The present invention is directed to a method of treating and/or managing cystic fibrosis in a patient by administering an electrolyzed saline solution to the patient.

### BACKGROUND OF THE INVENTION

Cystic fibrosis (CF) is a genetic disease affecting approximately 30,000 children and adults in the United States. A gene mutation causes the body to produce abnormally thick mucus that builds up in the lungs and leads to life-threatening lung infections. These thick secretions also obstruct the pancreas, preventing digestive enzymes from reaching the intestines to help break down and absorb food. The mucus also can block the bile duct in the liver, eventually causing permanent liver damage in approximately six percent of people with CF

Clearing mucus from the lungs is an important part of the daily CF treatment regimen. Chest physical therapy is a form of airway clearance done by vigorous clapping on the back and chest to dislodge the thick mucus from the lungs. Other types of treatments include TOBI® (tobramycin solution for inhalation), an aerosolized antibiotic used to treat lung infections; Pulmozyme®, a mucus-thinning drug shown to reduce the number of lung infections and improve lung function; and azithromycin, an antibiotic recently proven to be effective in people with CF whose lungs are chronically infected with the common Pseudomonas aeruginosa bacteria. The repeated use of antibiotics, however, raises concern of antibiotic resistance. Further, many of the current treatments may results in intolerable side effects. Therefore, there is a need for a safe, alternative therapy for treating and/or managing patients with cystic fibrosis.

### SUMMARY OF THE INVENTION

The present invention provides methods of treating and/or managing cystic fibrosis by administering an electrolyzed saline solution. In an embodiment, the electrolyzed saline solution has a pH between 5.5 and 6.2 and an available free chlorine content between about 50 and 1000 ppm when produced by an electrolytic cell. In a preferred embodiment, the solution is a hypertonic solution.

The present invention also provides a method of irrigating the gastrointestinal tract by rectally administering an electrolyzed saline solution having a pH between about 5 and 7 and an available free chlorine content between about 50 and 1000 ppm when produced by an electrolytic cell.

In another embodiment, the present invention provides a method of treating and/or managing cystic fibrosis in a patient comprising administering to the patient an electrolyzed saline solution having a pH between about 5.0 and 7.0 and an available free chlorine content between about 50 and 1000 parts per million (ppm) when produced by an electrolytic cell. The method further comprises administering nitric oxide to the patient.

In yet another embodiment, the present invention provides a method of treating and/or managing cystic fibrosis in a patient comprising administering to the patient via a fogger, vaporizer, or a humidifier an electrolyzed saline solution having a pH between about 5.0 and 7.0 and an available free chlorine content between about 50 and 1000 parts per million (ppm) when produced by an electrolytic cell.

### DETAILED DESCRIPTION OF THE INVENTION

The present invention provides a method of treating and/or managing cystic fibrosis in a patient by administering an electrolyzed saline solution to the patient. By “treating” or “managing” is meant improving, preventing the worsening of, and/or alleviating the symptoms of cystic fibrosis. Specifically, treating and/or managing cystic fibrosis can include any one or more of: (1) improving lung function; (2) improving quality of life; (3) reducing pulmonary exacerbation; (4) reducing the microbial load in the lungs; (5) irrigating the gastrointestinal tract; and treating other mucus membranes in the body.

Lung function can be improved by improving the forced expiratory volume in one second (FEV₁), the forced vital capacity (FVC), the forced expiratory flow between 25 and 75% FVC (FEF₂₅₋₇₅), the residual volume as a proportion of total lung capacity (RV:TLC), and/or the whole-lung mucus clearance. Lung function can be measured by spirometry, plethysmography, and/or quantitative air trapping. Lung function can also be assessed by measuring lung volume according to American Thoracic Society standards as described by the American Thoracic Society in “Standardization of spirometry,” 1994 update, Am. J. Respir. Crit Care Med 1995; 152: 1107-36, which is incorporated by reference herein. To determine mucus clearance, the amount of particles cleared from the whole lung can be compared to the baseline amount of particles in the whole lung. For example, the ratio of particle deposition in central regions of the lung can be compared with peripheral regions (C:P ratio).

Improving the quality of life of the patient can be measured by the Quittner & Sweeny Cystic Fibrosis Questionaire (domain scores 75 or better), as described by Quittner A L, Sweeny S, Watrous M, et al., “Translation and linguistic validation of a disease-specific quality of life measure for cystic fibrosis,” J. Pediatr. Psychol. 2000; 25: 403-14,which is incorporated by reference herein, or the Medical Outcomes Study 36-item Short-Form General Health Survey (SF-36) (score of 75 or better), as described in Ware, J E Jr, Sherbourne C D. “The MOS 36-item short-form health survey (SF-36). I. Conceptual framework and item selection.” Med. Care 1992; 30: 4783-49, which is incorporated by reference herein.

Pulmonary exacerbation can be determined by the clinical need for intravenous antibiotics as indicated, by the presence of, for example, at least four to twelve of the following signs or symptoms: a change in sputum volume or color, new or increased hemoptysis, increased cough, increased dyspnea, malaise, fatigue, or lethargy, a temperature above 38° C., anorexia or weight loss, sinus pain or tenderness, a change in sinus discharge, a change in findings on physical examination of the chest, a decrease in pulmonary function by 10% or more from a previously recorded value, or a radiographic change indicative of pulmonary infection. Alternatively, pulmonary exacerbation can be determined by the presence of, for example, at least four of the twelve aforementioned symptoms or signs, regardless of whether any antibiotics are given. Any improvements in lung function, quality of life or pulmonary exacerbation are measured from the patient's baseline condition prior to administering an electrolyzed saline solution of the present invention.

Regarding reducing the microbial load in the lungs, an electrolyzed saline solution can result in a reduction in the bacterial, fungal, and/or viral load in the lungs. For example, an electrolyzed saline solution can result in a reduction in the concentration of any one or more of Pseudomonas aeruginosa, Staphylococcus aureus, B. cepacia, Stenotrophomonas maltophilia, Candida albicans, non-Candidia albicans, aspergillus species, and Haemophilus influenzae. Such organisms can be identified in the sputum of the patient using standard microbiologic techniques including the API 20 NE system (BioMerieux Vitek). Quantification of pathogens can be performed using the modifications described by Wong, K, Roberts D S, Owens L, Fife M, Smith A L, “Selective media for the quantitation of bacteria in cystic fibrosis sputum. J Med Microbiol. 1984; 17: 113-9, which is incorporated by reference herein. Preferably, an electrolyzed saline solution of the present invention hydrolyzes DNA in the sputum of the patient, breaks down biofilm and its polysaccharide protein matrix components, and/or neutralize endotoxins produced by biofilms thereby reducing viscoelasticity of mucus.

Regarding irrigating the gastrointestinal tract, an electrolyzed saline solution of the present invention can be administered to treat, for example, constipation or diarrhea. In such embodiments, an electrolyzed saline solution can be administered rectally, for example, by an enema, flush or lavage.

Regarding treating other mucus membranes, such membranes include any of the mucus membranes in the body including in the eye, ears, gastrointestinal tract, nose, sinuses, throat and/or lungs. Such treating can result, for example, in a reduction in the inflammation of the membranes, reduced microbial count in the membranes, and/or clearance or reduction of mucus lining such membranes.

An electrolyzed saline solution, according to the present invention, is a biocidal, mucolytic output solution produced via electrolysis in an electrochemical cell and contains a mixture of active species, including predominantly hypohalous acid (HOX), wherein X is a halide. The halide is an anion such as fluoride (F⁻), chloride (Cl⁻), bromide (Br⁻), iodide (I⁻) or astatide (At⁻). In a preferred embodiment, the solution contains predominantly hypochlorous acid. In certain embodiments, an electrolyzed saline solution comprises at least 90% hypohalous acid and preferably at least 95% hypohalous acid. In an embodiment, an electrolyzed saline solution has an available free chlorine content (AFC) between about 50 and 1000 parts per million (ppm). Preferably, the solution has an AFC concentration between about 50 and 500, more preferably between about 50 and 250, more preferably between about 50 and 200, even more preferably between about 50 and 150 and even more preferably between about 50 and 100. Such AFC concentration refers to the AFC concentration of the solution after electrolysis but before nebulization or atomization.

In an embodiment, an electrolyzed saline solution of the present invention has a pH between about 5 and 7, including all intermediate values therebetween. Preferably, the solution has a pH between about 5.0 and 6.2, more preferably between about 5.2 and 6.0, even more preferably between about 5.2 and 5.8 and even more preferably between about 5.4 and 5.5. Such preferable pH ranges include all intermediate values therebetween. Further, such preferable pH ranges result in high purity HOX, with low concentrations of toxic/cytotoxic agents such as hypochlorite (OCI⁻) and chlorine (Cl2). In preferred embodiments, a pH range of 5.0 to 6.2 results in less than or equal to 20% hypochlorite, more preferably less than or equal to 15% hypochlorite, even more preferably less than or equal to 10% hypochlorite, and even more preferably less than or equal to 5% hypochlorite. Alternatively or in addition, such a pH range preferably results in a chlorine concentration of less than or equal to 5%, more preferably less than or equal to 3%, even more preferably less than or equal to 1% and even more preferably about 0%.

An electrolyzed saline solution of the present invention can be hypertonic, isotonic or hypotonic. In a preferred embodiment, the saline solution is hypertonic (i.e. has a salt concentration greater than 9 g/L). In a more preferred embodiment, the solution has a salt concentration between about 70 g/L and 90 g/L.

An electrolyzed saline solution of the present invention can be administered to the patient via several different routes of administration, such as orally, via inhalation, topically, rectally, vaginally, intraperitoneally, subcutaneously, and/or intraveneously. The route of administration may be dictated by the mode of treatment. For example, if it is desired to treat the mucus membranes in the eye, an electrolyzed saline solution can be applied via an eye wash or eye drops; if treating the ears, a solution can be applied by ear drops or irrigation; if treating the gastrointestinal tract, an electrolyzed saline solution can be applied by an enema or irrigation; if treating the nose and sinuses, a solution can be applied by pulsatile irrigation, lavage, nasal drops, or nasal inhalation; if treating the throat, a solution can be applied via an oral rinse or gargle; if treating the lungs, an electrolyzed saline solution be administered via a humidifier, vaporizer, fogger or similar device. Of course, other methods of administration for treating these particular sites in the body or other sites may be utilized and the above methods are only exemplary.

In a preferred embodiment, an electrolyzed saline solution is administered via inhalation. For such a route of administration, an electrolyzed saline solution may contain any of a variety of known aerosol propellants, such as dichlorodifluoromethane, propane, nitrogen, and the like, useful for endopulmonary and/or intranasal inhalation administration. In addition, a variety of co-solvents, surfactants, stabilizers (e.g., antioxidants, chelating agents, inert gases and buffers) may also be present. An electrolyzed saline solution can be administered by inhalation using any aerosolization technique, including but not limited to, standard nebulization or electrohydrodynamic aerosolization. For example, the solution can be delivered using a nebulizer or compressor known in the art, such as the Pari LC Star nebulizer, the Pan LC Plus nebulizer, the Pari Pronbe Ultra compressor (all available from Pari), a DeVilbiss 646 nebulizer, or a DeVilbiss Pulmo-Aide air compressor. Of course, other types of nebulizers, compressors or aerosolization devices can be used and the above-mentioned devices are only exemplary. In certain embodiments, an electrolyzed saline solution is administered via or in conjunction with a humidifier, vaporizer, fogger or similar device. In an even more preferred embodiment, an electrolyzed saline solution is administered via a combination device comprising a humidifier, vaporizer or fogger connected to an electrochemical cell. In such embodiments, a humidifier, vaporizer, fogger or similar device can be particularly useful in home use where the device is placed in the same space as the patient as well as the patient's caregiver to protect both parties.

Suitable dosages of an electrolyzed saline solution of the present invention can be determined by a physician. or qualified medical professional depending on factors such as the nature and severity of the illness, the route and frequency of administration, the duration of treatment, the condition of the patient, the size and age of the patient, and other factors. One of skill in the art would also know how to monitor the progress of the treatment in order to determine an effective dose. For example, one skilled in the art could monitor spirometries, chest X-rays, sputum cultures and blood tests. An electrolyzed saline solution may be administered as frequently as necessary in order to obtain the desired therapeutic effect of treating the cystic fibrosis. Frequency of administration will depend, for example, on the nature of the dosage form used and the severity of the cystic fibrosis being treated. For example, if administering in the eye or ear, 1 to 2 drops of an electrolyzed saline solution can be administered 1 to 10 times per day. In another embodiment, the solution can be administered in the eye or ear 1 to 4 times per day. For chronic use, one to two drops of the solution can be administered once to twice daily in certain embodiments. In a non-limiting example, five milliliters of an electrolyzed saline solution is administered once daily, twice daily, or every other day alone or in combination with other therapies as described in more detail below.

An electrolyzed saline solution of the present invention can be administered to any animal, such as a mammal. In one embodiment, the mammal is a human. In other embodiments, an electrolyzed saline solution is used in a veterinary application for administration to mammals, reptiles, or birds. Non-limiting examples of animals include cows, chickens, pig, dogs, cats, or rodents. In certain embodiments, the mammal is an animal suitable for serving as food to a human or another animal.

In certain embodiments, an electrolyzed saline solution is administered in combination with other therapeutic agents. Non-limiting examples of such therapeutic agents include bronchodilators include albuterol; DNases and other mucolytic therapies such as deoxyribonuclease (Domase alfa) and purinergic compounds such as P2Y2 receptor activators including, for example, Diquafosol; amiloride and UTP; vitamins such as, for example, vitamin A, D, E, K, and C; antioxidants such as glutathione; carotenoids such as, for example, acylyl-carnitine; nitric oxide; pancreatic enzymes/digestive enzymes for GI irrigation; hypertonic saline such as 7-12% saline and preferably 7% saline; isotonic saline; hypotonic saline, preferably 0.3-0.45% concentration; and any combination thereof.

Such additional therapeutic agents can be administered before, after or concurrently with administration of an electrolyzed saline solution. In a non-limiting example, an additional therapeutic agent is administered before, after or concurrently with administration of an electrolyzed saline solution on the same day. Alternatively, an electrolyzed saline solution and an additional therapeutic agent are administered on alternate days. For certain therapeutic agents certain relative time periods of administration may be preferred. For example, in the case of an antioxidant, it is preferably administered after administration of an electrolyzed saline solution to react with the hypohalous acid to prevent any undesired oxidizing effects of the hypohalous acid after the hypohalous acid has acted on the microbes in the mucosal membrane(s) of the patient. In the case of a bronchodilator, it is preferably administered before each inhalation of the electrolyzed saline solution. Particular doses of any of these and other additional therapeutic agents can be determined by a physician or qualified medical professional depending on factors such as the type of therapeutic agent, nature and severity of the illness, the route and frequency of administration, the duration of treatment, the condition of the patient, the size and age of the patient, and other factors. One of skill in the art would also know how to monitor the progress of the treatment in order to determine an effective dose as described above. In certain embodiments where the additional therapeutic agent is inhaled nitric oxide, an exemplary dose is less than 100 ppm and more preferably less than 50 ppm. In neonatals, an exemplary dose is 20 ppm or less.

An electrolyzed saline solution can be prepared by any suitable method that results in the electrolyzed saline solution having the above-described characteristics. In a preferred embodiment, the electrolyzed saline solution is manufactured according to the methods and devices described in U.S. patent application No. 20060278585 or U.S. Pat. No. 6,632,347,both of which are incorporated by reference herein. Preferably, an electrolyzed saline solution is produced onsite and on demand at a patient's home, healthcare facility, hospital and pharmacy. Methods for such on-site and on demand production are described in U.S. patent application No. 20060278585. In a preferred embodiment, an electrolyzed saline solution is produced by a electrochemical generator, preferably suitable for home use, that is connected to a vaporizer, humidifier or fogger for delivery of the electrolyzed saline. solution. Additional therapeutic agents can also be delivered by the vaporizer, humidifier or flogger.

The foregoing description has been set forth merely to illustrate the invention and are not intended as being limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

We claim:

1. A method of treating and/or managing cystic fibrosis in a patient comprising:
administering to the patient an electrolyzed saline solution having a pH between 5.0 and 6.2 and an available free chlorine content between about 50 and 1000 parts per million (ppm) when produced by an electrolytic cell.

2. The method of claim 1, wherein the electrolyzed saline solution has a pH between 5.2 and 6.0.

3. The method of claim 1, wherein the electrolyzed saline solution has a pH between 5.2 and 5.8.

4. The method of claim 1, wherein the electrolyzed saline solution has a pH of 5.4.

5. The method of claim 1, wherein the electrolyzed saline solution has a pH of 5.5.

6. The method of claim 1, wherein the electrolyzed saline solution is a hypertonic solution.

7. The method of claim 1, wherein treating and/or managing the cystic fibrosis comprises improving lung function in the patient.

8. The method of claim 1, wherein treating and/or managing the cystic fibrosis comprises improving the quality of life of the patient.

9. The method of claim 1, wherein treating and/or managing the cystic fibrosis comprises decreasing the pulmonary exacerbation of the patient.

10. The method of claim 1, wherein treating and/or managing the cystic fibrosis comprises decreasing the microbial load in the lungs of the patient.

11. The method of claim 1, wherein treating and/or managing the cystic fibrosis comprises irrigating the gastrointestinal tract of the patient.

12. The method of claim 1, wherein treating and/or managing cystic fibrosis comprises treating the mucus membranes of the patient.

13. The method of claim 1, further comprising administering vitamins to the patient.

14. The method of claim 1, further comprising administering an antioxidant to the patient after administration of the electrolyzed saline solution.

15. A method of treating and/or managing cystic fibrosis in a patient comprising:
irrigating the gastrointestinal tract of the patient by rectally administering to the patient an electrolyzed saline solution having a pH between about 5.0 and 7.0 and an available free chlorine content between about 50 and 1000 parts per million (ppm) when produced by an electrolytic cell.

16. The method of claim 13, wherein the electrolyzed saline solution has a pH of 5.4.

17. A method of treating and/or managing cystic fibrosis in a patient comprising:
administering to the patient an electrolyzed saline solution having a pH between about 5.0 and 7.0 and an available free chlorine content between about 50 and 1000 parts per million (ppm) when produced by an electrolytic cell; and
administering nitric oxide to the patient.

18. A method of treating and/or managing cystic fibrosis in a patient comprising:
administering to the patient via a fogger, vaporizer, or a humidifier an electrolyzed saline solution having a pH between about 5.0 and 7.0 and an available free chlorine content between about 50 and 1000 parts per million (ppm) when produced by an electrolytic cell.

19. The method of claim 18, wherein the electrolyzed saline solution is administered via a flogger.

20. The method of claim 18, wherein the electrolyzed saline solution is administered via a humidifier.

21. The method of claim 18, wherein the electrolyzed saline is administered via a vaporizer.
